(19) United States
(12) Patent Application Publication
(10) Pub. No.: US 2023/0260651 A1
(43) Pub. Date: Aug. 17, 2023

(54) METHOD, ELECTRONIC SYSTEM, AND COMPUTER PROGRAM PRODUCT FOR ESTABLISHING DECISION TREE FOR DISEASE PREDICTION

(71) Applicant: Wistron Corp., New Taipei (TW)

(72) Inventor: Li-Wen LO, New Taipei City (TW)

(21) Appl. No.: 17/846,957

(22) Filed: Jun. 22, 2022

(30) Foreign Application Priority Data
Feb. 17, 2022 (TW) 111105739

(51) Int. Cl. G16H 50/20

(52) U.S. Cl. CPC G16H 50/20

(57) ABSTRACT

A method for establishing a decision tree for disease prediction is provided. The method receives a plurality of physiological measurement data corresponding to different diseases. The method classifies the physiological measurement data corresponding to the purpose. The method calculates at least one cutting point of the physiological measurement data. The method branches the decision tree corresponding to the at least one cutting point. The method prunes the decision tree to complete the establishment of the decision tree. The present invention can assist doctors in medical diagnosis, give preventive medication in advance based on the prediction results, and calculate the data of each terminal branch of the decision tree to obtain the probability of a single disease, which can improve the accuracy of more disease predictions.

### CROSS REFERENCE TO RELATED APPLICATIONS

This application claims priority to and the benefit of Taiwan Application No. 111105739, filed on Feb. 17, 2022, the entirety of which is incorporated by reference herein.

### FIELD OF THE DISCLOSURE

The disclosure is related to a method of assisting in disease prediction, and in particular, it is related to a method, an electronic system, and a computer program product for establishing a decision tree for disease prediction.

### DESCRIPTION OF THE RELATED ART

At present, doctors must rely on their experience to determine whether a patient suffers from a particular disease, and then they conduct further examinations such as blood work or computed tomography. However, most doctors will let patients take painkillers or anti-inflammatory drugs to address their symptoms, which may increase the chance of delaying medical treatment. Therefore, it is necessary to import Fast Healthcare Interoperability Resources (FHIR), a common international format, so that patients can provide complete medical records to hospitals in various places, reducing the chances of misdiagnosis and gaining valuable treatment time.

Now, more and more medical institutions are introducing artificial intelligence to help judge images, reducing the burden on pathologists and increasing the possibility of early detection of diseases. In the case of young doctors with less experience, artificial intelligence can also be used to assist in disease recognition, reducing the possibility of misdiagnosis during experiential learning. Therefore, how to establish a mechanism to assist in disease prediction has become an important issue.

### BRIEF SUMMARY OF THE DISCLOSURE

In order to resolve the issue described above, the present disclosure provides a method for establishing a decision tree for disease prediction. The method includes the following steps. A plurality of physiological measurement data corresponding to different diseases is received. The physiological measurement data classified by the purpose. At least one cutting point of the physiological measurement data is calculated. The decision tree is branched at the cutting point. The decision tree is pruned, completing the establishment of the decision tree.

According to the method disclosed above, the step of calculating the cutting point of the physiological measurement data includes calculating the value of the cutting point of the physiological measurement data by using the specific function associated with the physiological measurement data and the absolute value of the correlation coefficient that is associated with the physiological measurement data.

According to the method disclosed above, the step of branching the decision tree corresponding to the cutting point includes setting the cutting point with the smallest value as a branch node of the decision tree; and determining whether the step of branching can be continued or not.

According to the method disclosed above, the step of pruning the decision tree to complete the establishment of the decision tree includes pruning the decision tree by using the Akaike Information Criterion (AIC).

According to the method disclosed above, the step of classifying the physiological measurement data corresponding to the purpose includes classifying the physiological measurement data as classification data when the physiological measurement data are used for the estimation of the probability of occurrence of different diseases.

According to the method disclosed above, when the physiological measurement data are classified as the classification data, the specific function is a Gini coefficient formula; the Gini coefficient formula is as follows: Gini(D)=Σᵢ₌₁ⁿp(xᵢ)×(1−p(xᵢ))=1−Σᵢ₌₁ⁿp(xᵢ)²; wherein xᵢ is the data corresponding to a disease among the physiological measurement data; p(xᵢ) is the probability of occurrence of the data corresponding to the disease among the physiological measurement data; and n is the number of disease types corresponding to the physiological measurement data.

According to the method disclosed above, the correlation coefficient is as follows:

$$r ( i ) = \frac{{\sum }_{j = 1}^{n} ( {x}_{j} - \overline{x} ) ( {y}_{j} - \overline{y} )}{\sqrt{{\sum }_{j = 1}^{n} {( {x}_{j} - \overline{x} )}^{2}} \sqrt{{\sum }_{j = 1}^{n} {( {y}_{j} - \overline{y} )}^{2}}} ;$$

wherein i is one of the physiological measurement data; n is the number of physiological measurement data; xⱼ is an independent variable and represents the physiological measurement data; x̅ is the mean of independent variables and represents the mean of the physiological measurement data; yⱼ is a dependent variable and represents the value corresponding to a disease; and y̅ is the mean of dependent variables and represents the mean of the value corresponding to the disease.

According to the method disclosed above, the physiological measurement data comprises gender, Body Mass Index (BMI), uric acid, total cholesterol, white blood cells, and blood sugar.

According to the method disclosed above, the value of the cutting point of the physiological measurement data is equal to Gini(D)×|r(i)|.

According to the method disclosed above, the AIC is as follows: AIC=−2×1+2×(k+1); wherein 1 is a likelihood function, and k is the number of parameters.

According to the method disclosed above, the method further includes calculating the correct rate of each terminal branch of the decision tree corresponding to the different diseases.

According to the method disclosed above, in response to determine whether the step of branching can be continued or not, the method includes repeating the step of calculating the value of the cutting point of the physiological measurement data and the step of setting the cutting point with the smallest value as the branch node of the decision tree, until the step of branching cannot be continued; or repeating the step of calculating the value of the cutting point of the physiological measurement data and the step of setting the cutting point with the smallest value as the branch node of the decision tree, until the number of physiological measurement data included in the branch node is less than or equal to a preset number of physiological measurement data corresponding to each disease.

According to the method disclosed above, in response to determine whether the step of branching can be continued or not, the method includes sorting the physiological measurement data according to gender from female to male; sorting the physiological measurement data according to BMI from low to high; sorting the physiological measurement data according to uric acid from low to high; sorting the physiological measurement data according to total cholesterol from least to most; sorting the physiological measurement data according to the number of white blood cells from least to most; and sorting the physiological measurement data according to blood sugar from low to high.

According to the method disclosed above, in response to determine whether the step of branching can be continued or not, the method includes calculating the product between the specific function and the absolute value of the correlation coefficient according to the results of sorting by gender, BMI, uric acid, total cholesterol, white blood cells, and blood sugar of the physiological measurement data.

The present disclosure also provides an electronic system to establish a decision tree for disease prediction. The electronic system includes a first processor, a data base and a second processor. The first processor is configured to receive a plurality of physiological measurement data corresponding to different diseases from a hospital. The data base is configured to store the physiological measurement data. The second processor is configured to obtain the physiological measurement data from the data base to execute the following steps. The steps include classifying the physiological measurement data corresponding to the purpose; calculating at least one cutting point of the physiological measurement data; branching the decision tree corresponding to the cutting point; and pruning the decision tree to complete the establishment of the decision tree.

According to the electronic system disclosed above, the second processor's calculation of the cutting point of the physiological measurement data includes calculating the value of the cutting point of the physiological measurement data by using the specific function that is associated with the physiological measurement data and the absolute value of the correlation coefficient that is associated with the physiological measurement data. This is performed by the second processor.

According to the electronic system disclosed above, when the physiological measurement data are used for the estimation of the probability of occurrence of different diseases, the second processor classifies the physiological measurement data as classification data.

According to the electronic system disclosed above, when the second processor classifies the physiological measurement data as the classification data, the specific function is a Gini coefficient formula; the Gini coefficient formula is as follows: Gini(D)=Σᵢ₌₁ⁿp(xᵢ)×(1−p(xᵢ))=1−Σᵢ₌₁ⁿp(xᵢ)²; wherein xᵢ is the data corresponding to a disease among the physiological measurement data; p(xᵢ) is the probability of occurrence of the data corresponding to the disease among the physiological measurement data; and n is the number of disease types corresponding to the physiological measurement data.

According to the electronic system disclosed above, the correlation coefficient is as follows:

$$r ( i ) = \frac{{\sum }_{j = 1}^{n} ( {x}_{j} - \overline{x} ) ( {y}_{j} - \overline{y} )}{\sqrt{{\sum }_{j = 1}^{n} {( {x}_{j} - \overline{x} )}^{2}} \sqrt{{\sum }_{j = 1}^{n} {( {y}_{j} - \overline{y} )}^{2}}} ;$$

wherein i is one of the physiological measurement data; n is the number of physiological measurement data; xⱼ is an independent variable and represents the physiological measurement data; x̅ is the mean of independent variables and represents the mean of the physiological measurement data; yⱼ is a dependent variable and represents the value corresponding to a disease; and y̅ is the mean of dependent variables and represents the mean of the value corresponding to the disease.

According to the electronic system disclosed above, the value of the cutting point of the physiological measurement data is equal to Gini(D)×|r(i)|.

The present disclosure also provides a computer program product to establish a decision tree for disease prediction. The computer program product is applied to an electronic system having a first processor, a second processor, and a data base. The computer program product includes a receiving instruction, a storing instruction, a reading instruction, a classifying instruction, a calculating instruction, a branching instruction, and a pruning instruction. The receiving instruction enables the first processor to receive a plurality of physiological measurement data corresponding to different diseases from a hospital. The storing instruction enables the data base to store the physiological measurement data. The reading instruction enables the second processor to obtain the physiological measurement data from the data base. The classifying instruction enables the second processor to classify the physiological measurement data corresponding to the purpose. The calculating instruction enables the second processor to calculate at least one cutting point of the physiological measurement data. The branching instruction enables the second processor to branch the decision tree corresponding to the cutting point. The pruning instruction enables the second processor to prune the decision tree. After the first processor finishes the receiving instruction, the data base finishes the storing instruction, and the second processor finishes the reading instruction, the classifying instruction, the calculating instruction, the branching instruction, and the pruning instruction, the establishment of the decision tree is completed.

### BRIEF DESCRIPTION OF THE DRAWINGS

The disclosure can be more fully understood by reading the subsequent detailed description with references made to the accompanying figures. It should be understood that the figures are not drawn to scale in accordance with standard practice in the industry. In fact, it is allowed to arbitrarily enlarge or reduce the size of components for clear illustration. This means that many specific details, relationships and methods are disclosed to provide a complete understanding of the disclosure.

FIG. 1 is a flow chart of a method for establishing a decision tree for disease prediction in accordance with some embodiments of the disclosure.

FIG. 2 is a schematic diagram of the decision tree in accordance with some embodiments of the disclosure.

FIG. 3 is a schematic diagram of an electronic system to establish a decision tree for disease prediction in accordance with some embodiments of the disclosure.

### DETAILED DESCRIPTION OF THE DISCLOSURE

In order to make the above purposes, features, and advantages of some embodiments of the present disclosure more comprehensible, the following is a detailed description in conjunction with the accompanying drawings.

It should be understood that the words “comprise” and “include” used in the present disclosure are used to indicate the existence of specific technical features, values, method steps, operations, units and/or components. However, it does not exclude that more technical features, values, method steps, work processes, units, components, or any combination of the above can be added.

The words “first”, “second”, “third”, and “fourth” are used to describe components, they are not used to indicate the priority order of or advance relationship, but only to distinguish components with the same name.

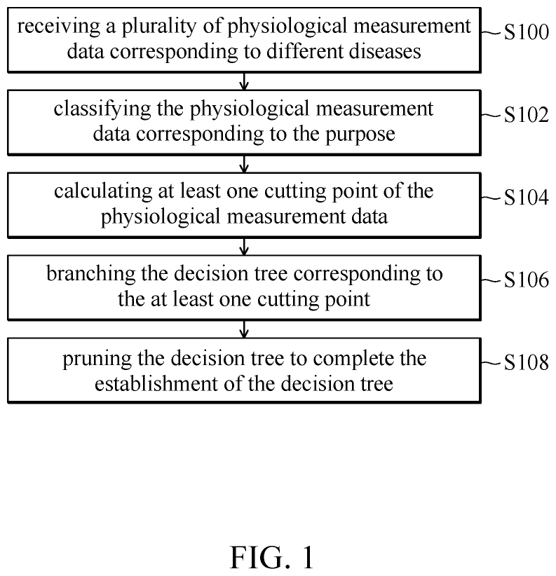

FIG. 1 is a flow chart of a method for establishing a decision tree for disease prediction in accordance with some embodiments of the disclosure. As shown in FIG. 1, the method for establishing the decision tree for disease prediction of the present disclosure includes: receiving a plurality of physiological measurement data corresponding to different diseases (step S100); classifying the physiological measurement data corresponding to the purpose (step S102); calculating at least one cutting point of the physiological measurement data (step S104); branching the decision tree corresponding to the cutting point (step S106); and pruning the decision tree to complete the establishment of the decision tree (step S108).

In detail, in step S104, the method for establishing the decision tree for disease prediction of the present disclosure further includes the following step: The value of the cutting point of the physiological measurement data is calculated by using a specific function associated with the physiological measurement data and the absolute value of the correlation coefficient associated with the physiological measurement data. In step S106, the method for establishing the decision tree for disease prediction of the present disclosure further includes the following steps. The cutting point with the smallest value is set as a branch node in the decision tree. A determination is made as to whether the step of branching can be continued or not. In step S108, the method for establishing the decision tree for disease prediction of the present disclosure further includes pruning the decision tree using the Akaike Information Criterion (AIC).

In some embodiments, the decision tree established in the present disclosure for disease prediction is a Classification and Correlation Coefficient Regression Trees (CCRT) decision tree. The CCRT decision tree is an improved version of the traditional and well-known Classification and Regression Trees (CART) decision tree. The correlation coefficient is added into the CCRT decision tree to adjust the parameters to improve the disease prediction ability of the CCRT decision tree. In step S100, the physiological measurement data are the medical record data of each patient from the hospital. For example, a patient's medical record data may include gender, Body Mass Index (BMI), uric acid, total cholesterol, white blood cells, and blood sugar, but the present disclosure is not limited thereto.

Table 1 is physiological measurement data corresponding to different diseases of five patients from the hospital. The physiological measurement data in Table 1 are provided as examples.

TABLE 1

| | | | | Total | white | | |
|---|---|---|---|---|---|---|---|
| Data | | | Uric | choles- | blood | blood | |
| number | Gender | BMI | acid | terol | cells | sugar | disease |
| 1 | F | 18 | 7.3 | 150 | 15.3 | 201 | Diabetes |
| 2 | F | 36 | 9.8 | 285 | 20.8 | 125 | Atherosclerosis |
| 3 | M | 32 | 6.5 | 201 | 8.51 | 100 | Hypertension |
| 4 | M | 24 | 5.7 | 187 | 4.38 | 131 | Fatty liver |
| 5 | M | 28 | 7.4 | 235 | 18.1 | 185 | Diabetes |

As shown in Table 1, patient No. 1 is a female, her BMI is 18, uric acid is 7.3, total cholesterol is 150, white blood cells is 15.3, and blood sugar is 201, and the doctor judges that the disease that patient No. 1 suffers from is diabetes. Patient No. 2 is a female, her BMI is 36, uric acid is 9.8, total cholesterol is 285, white blood cells is 20.8, and blood sugar is 125, and the doctor judges that the disease that patient No. 2 suffers from is atherosclerosis. Patient No. 3 is a male, his BMI is 32, uric acid is 6.5, total cholesterol is 201, white blood cells is 8.51, and blood sugar is 100, and the doctor judges that the disease that patient No. 3 suffers from is hypertension. Patient No. 4 is a male, his BMI is 24, uric acid is 5.7, total cholesterol is 187, white blood cells is 4.38, and blood sugar is 131, and the doctor judges that the disease that patient No. 4 suffers from is fatty liver. Patient No. 5 is a male, his BMI is 28, uric acid is 7.4, total cholesterol is 235, white blood cells is 18.1, and blood sugar is 185, and the doctor judges that the disease that patient No. 5 suffers from is diabetes.

In step S102, the physiological measurement data are classified as classification data when the physiological measurement data are used for the estimation of the probability of occurrence of different diseases. In some embodiments, the physiological measurement data are classified as numerical data when the physiological measurement data from the hospital are used for classification of different diseases. The CCRT decision tree of the present disclosure can process both classification data and numerical data. In some embodiments, when the physiological measurement data is classified as classification data in step S102, the specific function associated with the physiological measurement data in step S104 is a Gini coefficient formula. In detail, the Gini coefficient formula is shown as Equation 1 below.

Gini(D)=Σᵢ₌₁ⁿp(xᵢ)×(1−p(xᵢ))=1−Σᵢ₌₁ⁿp(xᵢ)² Equation 1

xᵢ is the data corresponding to a disease among the physiological measurement data; p(xᵢ) is the probability of occurrence of the data corresponding to the disease among the physiological measurement data; and n is the number of disease types corresponding to the physiological measurement data.

In step S104, the correlation coefficient is shown as Equation 2.

$$r ( i ) = \frac{{\sum }_{j = 1}^{n} ( {x}_{j} - \overline{x} ) ( {y}_{j} - \overline{y} )}{\sqrt{{\sum }_{j = 1}^{n} {( {x}_{j} - \overline{x} )}^{2}} \sqrt{{\sum }_{j = 1}^{n} {( {y}_{j} - \overline{y} )}^{2}}} ; \qquad Equation 2$$

i is one of the physiological measurement data; n is the number of physiological measurement data; xⱼ is an independent variable and represents the physiological measurement data; x̅ is the mean of independent variables and represents the mean of the physiological measurement data; yⱼ is a dependent variable and represents the value corresponding to a disease; and y̅ is the mean of dependent variables and represents the mean of the value corresponding to the disease. In some embodiments, the method of the present disclosure can convert gender F into value 2, gender M into value 1, diabetes into value 1, atherosclerosis into value 2, hypertension into value 3, and fatty liver into value 4, but the present disclosure is not limited thereto.

In detail, in step S104, the value of the cutting point of the physiological measurement data is equal to Gini(D)×|r(i)|, which is Equation 3.

In some embodiments, before the method of the present disclosure calculates the product of the Gini coefficient formula, Gini(D), and the absolute value of the correlation coefficient, |r(i)|, the physiological measurement data are sorted according to gender from female to male, BMI from low to high, uric acid from low to high, total cholesterol from least to most, the number of white blood cells from least to most, and blood sugar from low to high. In some embodiments, the method of the present disclosure calculates the product between Gini coefficient formula, Gini(D), and the absolute value of the correlation coefficient, |r(i)|, to obtain the value of the cutting point of the physiological measurement data according the sorting of gender, BMI, uric acid, total cholesterol, white blood cells, and blood sugar in the physiological measurement data.

The physiological measurement data in Table 1 are exemplified. The method of the present disclosure sorts the data of patients No. 1 to 5 as (1, 2, 3, 4, 5) according to gender, that is, the sorting of gender of patients No. 1 to 5 is (F, F, M, M, M). After that, the method of present disclosure calculate the value of cutting point between male and female in the data of patients No. 1 to 5, as shown in Equation 4 below.

$$Gini ( D ) \times \vert r \vert = \{ \{ 1 - [ {( \frac{1}{2} )}^{2} + {( \frac{1}{2} )}^{2} ] \} \times \frac{2}{5} + \{ 1 - [ {( \frac{1}{3} )}^{2} + {( \frac{1}{3} )}^{2} + {( \frac{1}{3} )}^{2} ] \} \times \frac{3}{5} \} \times 1 = \frac{3}{5} = 0.6 \qquad Equation 4$$

The method of the present disclosure converts gender into values and substitutes the values into Equations 3, 2 and 1 according to the sorting of the physiological measurement data (F, F, M, M, M) by gender to obtain Equation 4. For example, in the method of present disclosure, after sorting the physiological measurement data according to gender, the cutting point is between the first two F and the last three M. The data of the first two F correspond to different diseases (e.g., diabetes and atherosclerosis, respectively), thus the probability is ½ each. The left branch is

$$1 - [ {( \frac{1}{2} )}^{2} + {( \frac{1}{2} )}^{2} ]$$

multiplying by ⅖ (2 of 5 data). Similarly, the data of the last three M correspond to different diseases (e.g., hypertension, fatty liver, and diabetes), thus the probability is ⅓ each. The right branch is

$$1 - [ {( \frac{1}{3} )}^{2} + {( \frac{1}{3} )}^{2} + {( \frac{1}{3} )}^{2} ]$$

multiplying by ⅗ (3 of 5 data). According to the result of Equation 4, it can be obtained that the value of the cutting point sorted by gender is 0.6.

Then, the method of the present disclosure sorts the data of patients No. 1 to 5 as (1, 4, 5, 3, 2) according to BMI, that is, the sorting of BMI of patients No. 1 to 5 is (18, 24, 28, 32, 36). The method of the present disclosure first calculates the first cutting point according to BMI, that is, the first cutting point for BMI<((18+24)/2), as shown in Equation 5 below.

$$Gini ( D ) \times \vert r \vert = \{ \{ 1 - [ {( \frac{1}{1} )}^{2} ] \} \times \frac{1}{5} + \{ 1 - [ {( \frac{1}{4} )}^{2} + {( \frac{1}{4} )}^{2} + {( \frac{1}{4} )}^{2} + {( \frac{1}{4} )}^{2} ] \} \times \frac{4}{5} \} \times 1 = \frac{3}{5} = 0.6 \qquad Equation 5$$

For example, after sorting the physiological measurement data according to BMI, the first cutting point is between 18 and 24. In the physiological measurement data whose BMI is 18 (e.g., patient No. 1), the disease it corresponds to is diabetes, thus the probability is 1/1. Therefore, the left branch of the first cutting point is

$$1 - [ {( \frac{1}{1} )}^{2} ]$$

multiplying by ⅕ (1 of 5 data). Similarly, in the physiological measurement data whose BMI are 24, 28, 32, and 36 (e.g., patients No. 2˜5), the diseases they correspond to are all different (such as atherosclerosis, hypertension, fatty liver, and diabetes), thus the probability is ¼ each. Therefore, the right branch is

$$1 - [ {( \frac{1}{4} )}^{2} + {( \frac{1}{4} )}^{2} + {( \frac{1}{4} )}^{2} + {( \frac{1}{4} )}^{2} ]$$

multiplying by ⅘ (4 of 5 data). According to the result of Equation 5, it can be obtained that the value of the first cutting point sorted by BMI is 0.6.

Then, the method of the present disclosure calculates the next cutting point according to BMI, that is, the second cutting point for BMI<((24+28)/2), as shown in Equation 6 below.

$$Gini ( D ) \times \vert r \vert = \text{} \{ \{ 1 - [ {( \frac{1}{2} )}^{2} + {( \frac{1}{2} )}^{2} ] \} \times \frac{2}{5} + \{ 1 - [ {( \frac{1}{3} )}^{2} + {( \frac{1}{3} )}^{2} + {( \frac{1}{3} )}^{2} ] \} \times \frac{3}{5} \} \times 1 = \frac{3}{5} = 0.6 \qquad Equation 6$$

For example, after sorting the physiological measurement data according to BMI, the second cutting point is between 24 and 28. In the physiological measurement data whose BMI are 18 and 24 (e.g., patients No. 1 and No. 4), the diseases they correspond to are diabetes and fatty liver, thus the probability is ½ each. Therefore, the left branch of the second cutting point is

$$1 - [ {( \frac{1}{2} )}^{2} + {( \frac{1}{2} )}^{2} ]$$

multiplying by ⅖ (2 of 5 data). Similarly, in the physiological measurement data whose BMI are 28, 32 and 36 (e.g., patients No. 2, No. 3 and No. 5), the diseases they correspond to are all different (such as atherosclerosis, hypertension, and diabetes), thus the probability is ⅓ each. Therefore, the right branch is

$$1 - [ {( \frac{1}{3} )}^{2} + {( \frac{1}{3} )}^{2} + {( \frac{1}{3} )}^{2} ]$$

multiplying by ⅗ (3 of 5 data). According to the result of Equation 6, it can be obtained that the value of the second cutting point sorted by BMI is 0.6.

Then, the method of the present disclosure calculates the next cutting point according to BMI, that is, the third cutting point for BMI<((28+32)/2), as shown in Equation 7 below.

$$Gini ( D ) \times \vert \text{} r \vert = \text{} \{ \{ 1 - [ {( \frac{2}{3} )}^{2} + {( \frac{1}{3} )}^{2} ] \} \times \frac{3}{5} + \{ 1 - [ {( \frac{1}{2} )}^{2} + {( \frac{1}{2} )}^{2} ] \} \times \frac{2}{5} \} \times 0.1147 = 0.054 \qquad Equation 7$$

For example, after sorting the physiological measurement data according to BMI, the third cutting point is between 28 and 32. In the physiological measurement data whose BMI are 18, 24 and 28 (e.g., patients No. 1, No. 4 and No. 5), the diseases they correspond to are diabetes and fatty liver, thus the probability of occurrence of diabetes is ⅔, and the probability of occurrence of fatty liver is ⅓. Therefore, the left branch of the third cutting point is

$$1 - [ {( \frac{2}{3} )}^{2} + {( \frac{1}{3} )}^{2} ]$$

multiplying by ⅗ (3 of 5 data). Similarly, in the physiological measurement data whose BMI are 32 and 36 (e.g., patients No. 2 and No. 3), the diseases they correspond to are all different (such as atherosclerosis and hypertension), thus the probability is ½ each. Therefore, the right branch is

$$1 - [ {( \frac{1}{2} )}^{2} + {( \frac{1}{2} )}^{2} ]$$

multiplying by ⅖ (2 of 5 data). According to the result of Equation 7, it can be obtained that the value of the third cutting point sorted by BMI is 0.054.

Then, the method of the present disclosure calculates the next cutting point according to BMI, that is, the fourth cutting point for BMI<((32+36)/2), as shown in Equation 8 below.

$$Gini ( D ) \times \vert \text{} r \vert = \text{} \{ \{ 1 - [ {( \frac{2}{4} )}^{2} + {( \frac{1}{4} )}^{2} + {( \frac{1}{4} )}^{2} ] \} \times \frac{4}{5} + \{ 1 - {( \frac{1}{1} )}^{2} \} \times \frac{1}{5} \} \times 0.3163 = 0.158 \qquad Equation 8$$

For example, after sorting the physiological measurement data according to BMI, the fourth cutting point is between 32 and 36. In the physiological measurement data whose BMI are 18, 24, 28 and 32 (e.g., patients No. 1, No. 3, No. 4 and No. 5), the diseases they correspond to are diabetes, hypertension, and fatty liver, thus the probability of occurrence of diabetes is 2/4, the probability of occurrence of hypertension is ¼, and the probability of occurrence of fatty liver is ¼. Therefore, the left branch of the fourth cutting point is

$$1 - [ {( \frac{2}{4} )}^{2} + {( \frac{1}{4} )}^{2} + {( \frac{1}{4} )}^{2} ]$$

multiplying by ⅘ (4 of 5 data). Similarly, in the physiological measurement data whose BMI is 36 (e.g., patient No. 2), the disease it correspond to is atherosclerosis, thus the probability is 1/1. Therefore, the right branch is

$$1 - {( \frac{1}{1} )}^{2}$$

multiplying by ⅕ (1 of 5 data). According to the result of Equation 8, it can be obtained that the value of the fourth cutting point sorted by BMI is 0.158.

Moreover, the method of the present disclosure sorts the data of patients No. 1 to 5 as (4, 3, 1, 5, 2) according to uric acid, that is, the sorting of uric acid of patients No. 1 to 5 is (5.7, 6.5, 7.3, 7.4, 9.8). The method of the present disclosure first calculates the first cutting point according to uric acid, that is, the first cutting point for uric acid<((5.7+6.5)/2), as shown in Equation 9 below.

$$Gini ( D ) \times \vert \text{} r \vert = \text{} \{ \{ 1 - [ {( \frac{1}{1} )}^{2} ] \} \times \frac{1}{5} + \{ 1 - [ {( \frac{2}{4} )}^{2} + {( \frac{1}{4} )}^{2} + {( \frac{1}{4} )}^{2} ] \} \times \frac{4}{5} \} \times 1 = 0.5 \qquad Equation 9$$

For example, after sorting the physiological measurement data according to uric acid, the first cutting point is between 5.7 and 6.5. In the physiological measurement data whose uric acid is 5.7 (e.g., patient No. 4), the disease it corresponds to is fatty liver, thus the probability is 1/1. Therefore, the left branch of the first cutting point is

$$1 - [ {( \frac{1}{1} )}^{2} ]$$

multiplying by ⅕(1 of 5 data). Similarly, in the physiological measurement data whose uric acid are 6.5, 7.3, 7.4 and 9.8 (e.g., patients No. 1˜3 and No. 5), the diseases they correspond to are diabetes, atherosclerosis, and hypertension, thus the probability of occurrence of diabetes is 2/4, the probability of occurrence of atherosclerosis is ¼, and the probability of occurrence of hypertension is ¼. Therefore, the right branch is 1−

$$[ {( \frac{2}{4} )}^{2} + {( \frac{1}{4} )}^{2} + {( \frac{1}{4} )}^{2} ]$$

multiplying by ⅘ (4 of 5 data). According to the result of Equation 9, it can be obtained that the value of the first cutting point sorted by uric acid is 0.5.

Then, the method of the present disclosure calculates the next cutting point according to uric acid, that is, the second cutting point for uric acid<((6.5+7.3)/2), as shown in Equation 10 below.

$$Gini ( D ) \times \vert \text{} r \vert = \text{} \{ \{ 1 - [ {( \frac{1}{2} )}^{2} + {( \frac{1}{2} )}^{2} ] \} \times \frac{2}{5} + \{ 1 - [ {( \frac{2}{3} )}^{2} + {( \frac{1}{3} )}^{2} ] \} \times \frac{3}{5} \} \times 1 = 0.4667 \qquad Equation 10$$

For example, after sorting the physiological measurement data according to uric acid, the second cutting point is between 6.5 and 7.3. In the physiological measurement data whose uric acid are 5.7 and 6.5 (e.g., patients No. 3 and No. 4), the diseases they correspond to are fatty liver and hypertension, thus the probability is ½ each. Therefore, the left branch of the second cutting point is

$$1 - [ {( \frac{1}{2} )}^{2} + {( \frac{1}{2} )}^{2} ]$$

multiplying by ⅖ (2 of 5 data). Similarly, in the physiological measurement data whose uric acid are 7.3, 7.4 and 9.8 (e.g., patients No. 1, No. 5, and No. 2), the diseases they correspond to are diabetes and atherosclerosis, thus the probability of occurrence of diabetes is ⅔, and the probability of occurrence of atherosclerosis is ⅓. Therefore, the right branch is

$$1 - [ {( \frac{2}{3} )}^{2} + {( \frac{1}{3} )}^{2} ]$$

multiplying by ⅗ (3 of 5 data). According to the result of Equation 10, it can be obtained that the value of the second cutting point sorted by uric acid is 0.4667.

Then, the method of the present disclosure calculates the next cutting point according to uric acid, that is, the third cutting point for uric acid<((7.3+7.4)/2), as shown in Equation 11 below.

$$Gini ( D ) \times \vert r \vert = \{ \{ 1 - [ {( \frac{1}{3} )}^{2} + {( \frac{1}{3} )}^{2} + {( \frac{1}{3} )}^{2} ] \} \times \frac{3}{5} + \{ 1 - [ {( \frac{1}{2} )}^{2} + {( \frac{1}{2} )}^{2} ] \} \times \frac{2}{5} \} \times 0.982 = 0.589 \qquad Equation 11$$

For example, after sorting the physiological measurement data according to uric acid, the third cutting point is between 7.3 and 7.4. In the physiological measurement data whose uric acid are 5.7, 6.5 and 7.3 (e.g., patients No. 4, No. 3 and No. 1), the diseases they correspond to are all different (such as fatty liver, hypertension, and diabetes), thus the probability is ⅓ each. Therefore, the left branch of the third cutting point is

$$1 - [ {( \frac{1}{3} )}^{2} + {( \frac{1}{3} )}^{2} + {( \frac{1}{3} )}^{2} ]$$

multiplying by ⅗ (3 of 5 data). Similarly, in the physiological measurement data whose uric acid are 7.4 and 9.8 (e.g., patients No. 5 and No. 2), the diseases they correspond to are different (such as diabetes and atherosclerosis), thus the probability is ½ each. Therefore, the right branch is

$$1 - [ {( \frac{1}{2} )}^{2} + {( \frac{1}{2} )}^{2} ]$$

multiplying by ⅖ (2 of 5 data). According to the result of Equation 11, it can be obtained that the value of the third cutting point sorted by uric acid is 0.589.

Then, the method of the present disclosure calculates the next cutting point according to uric acid, that is, the fourth cutting point for uric acid<((7.4+9.8)/2), as shown in Equation 12 below.

$$Gini ( D ) \times \vert r \vert = \{ \{ 1 - [ {( \frac{2}{4} )}^{2} + {( \frac{1}{4} )}^{2} + {( \frac{1}{4} )}^{2} ] \} \times \frac{4}{5} + \{ 1 - {( \frac{1}{1} )}^{2} \} \times \frac{1}{5} \} \times 0.9876 = 0.4938 \qquad Equation 12$$

For example, after sorting the physiological measurement data according to uric acid, the fourth cutting point is between 7.4 and 9.8. In the physiological measurement data whose uric acid are 5.7, 6.5, 7.3 and 7.4 (e.g., patients No. 4, No. 3, No. 1, and No. 5), the diseases they correspond to are fatty liver, hypertension, and diabetes, thus the probability of occurrence of fatty liver is ¼, the probability of occurrence of hypertension is ¼, and the probability of occurrence of diabetes is 2/4. Therefore, the left branch of the fourth cutting point is

$$1 - [ {( \frac{2}{4} )}^{2} + {( \frac{1}{4} )}^{2} + {( \frac{1}{4} )}^{2} ]$$

multiplying by ⅘ (4 of 5 data). Similarly, in the physiological measurement data whose uric acid is 9.8 (e.g. patient No. 2), the disease it corresponds to is atherosclerosis, thus the probability is 1/1. Therefore, the right branch is

$$1 - {( \frac{1}{1} )}^{2}$$

multiplying by ⅕ (1 of 5 data). According to the result of Equation 12, it can be obtained that the value of the fourth cutting point sorted by uric acid is 0.4938.

After that, the method of the present disclosure sorts the data of patients No. 1 to 5 as (1, 4, 3, 5, 2) according to total cholesterol, that is, the sorting of total cholesterol of patients No. 1 to 5 is (150, 187, 201, 235, 285). The method of the present disclosure first calculates the first cutting point according to total cholesterol, that is, the first cutting point for total cholesterol<((150+187)/2), as shown in Equation 13 below.

$$Gini ( D ) \times \vert r \vert = \{ \{ 1 - [ {( \frac{1}{1} )}^{2} ] \} \times \frac{1}{5} + \{ 1 - [ {( \frac{1}{4} )}^{2} + {( \frac{1}{4} )}^{2} + {( \frac{1}{4} )}^{2} + {( \frac{1}{4} )}^{2} ] \} \times \frac{4}{5} \} \times 1 = \frac{3}{5} = 0.6 \qquad Equation 13$$

For example, after sorting the physiological measurement data according to total cholesterol, the first cutting point is between 150 and 187. In the physiological measurement data whose total cholesterol is 150 (e.g., patient No. 1), the disease it corresponds to is diabetes, thus the probability is 1/1. Therefore, the left branch of the first cutting point is

$$1 - [ {( \frac{1}{1} )}^{2} ]$$

multiplying by ⅕ (1 of 5 data). Similarly, in the physiological measurement data whose total cholesterol are 187, 201, 235 and 285 (e.g. patients No. 2˜5), the diseases they correspond to are all different (such as atherosclerosis, hypertension, fatty liver, and diabetes, thus the probability is ¼ each. Therefore, the right branch is

$$1 - [ {( \frac{1}{4} )}^{2} + {( \frac{1}{4} )}^{2} + {( \frac{1}{4} )}^{2} + {( \frac{1}{4} )}^{2} ]$$

multiplying by ⅘ (4 of 5 data). According to the result of Equation 13, it can be obtained that the value of the first cutting point sorted by total cholesterol is 0.6.

Then, the method of the present disclosure calculates the next cutting point according to total cholesterol, that is, the second cutting point for total cholesterol<((187+201)/2), as shown in Equation 14 below.

$$Gini ( D ) \times \vert r \vert = \{ \{ 1 - [ {( \frac{1}{2} )}^{2} + {( \frac{1}{2} )}^{2} ] \} \times \frac{2}{5} + \{ 1 - [ {( \frac{1}{3} )}^{2} + {( \frac{1}{3} )}^{2} + {( \frac{1}{3} )}^{2} \} \times \frac{3}{5} \} \times 1 = \frac{3}{5} = 0.6 \qquad Equation 14$$

For example, after sorting the physiological measurement data according to total cholesterol, the second cutting point is between 287 and 201. In the physiological measurement data whose total cholesterol are 150 and 187 (e.g., patients No. 1 and No. 4), the diseases they correspond to are diabetes and fatty liver, thus the probability is ½ each. Therefore, the left branch of the second cutting point is

$$1 - [ {( \frac{1}{2} )}^{2} + {( \frac{1}{2} )}^{2} ]$$

multiplying by ⅖ (2 of 5 data). Similarly, in the physiological measurement data whose total cholesterol are 201, 235 and 285 (e.g., patients No. 2, No. 3 and No. 5), the diseases they correspond to are all different (such as atherosclerosis, hypertension and diabetes), thus the probability is ⅓ each. Therefore, the right branch is

$$1 - [ {( \frac{1}{3} )}^{2} + {( \frac{1}{3} )}^{2} + {( \frac{1}{3} )}^{2} ]$$

multiplying by ⅗ (3 of 5 data). According to the result of Equation 14, it can be obtained that the value of the second cutting point sorted by total cholesterol is 0.6.

Then, the method of the present disclosure calculates the next cutting point according to total cholesterol, that is, the third cutting point for total cholesterol <((201+235)/2), as shown in Equation 15 below.

$$Gini ( D ) \times \vert r \vert = \{ \{ 1 - [ {( \frac{1}{3} )}^{2} + {( \frac{1}{3} )}^{2} + {( \frac{1}{3} )}^{2} ] \} \times \frac{3}{5} + \{ 1 - [ {( \frac{1}{2} )}^{2} + {( \frac{1}{2} )}^{2} ] \} \times \frac{2}{5} \} \times 0.824 = 0.4944 \qquad Equation 15$$

For example, after sorting the physiological measurement data according to total cholesterol, the third cutting point is between 201 and 235. In the physiological measurement data whose total cholesterol are 150, 187 and 201 (e.g., patients No. 1, No. 4 and No. 3), the diseases they correspond to are all different (such as fatty liver, hypertension, and diabetes), thus the probability is ⅓ each. Therefore, the left branch of the third cutting point is

$$1 - [ {( \frac{1}{3} )}^{2} + {( \frac{1}{3} )}^{2} + {( \frac{1}{3} )}^{2} ]$$

multiplying by ⅗ (3 of 5 data). Similarly, in the physiological measurement data whose total cholesterol are 235 and 285 (e.g., patients No. 5 and No. 2), the diseases they correspond to are different (such as diabetes and atherosclerosis), thus the probability is ½ each. Therefore, the right branch is

$$1 - [ {( \frac{1}{2} )}^{2} + {( \frac{1}{2} )}^{2} ]$$

multiplying by ⅖ (2 of 5 data). According to the result of Equation 15, it can be obtained that the value of the third cutting point sorted by total cholesterol is 0.4944.

Then, the method of the present disclosure calculates the next cutting point according to total cholesterol, that is, the fourth cutting point for total cholesterol<((235+285)/2), as shown in Equation 16 below.

$$Gini ( D ) \times \vert r \vert = \{ \{ 1 - [ {( \frac{2}{4} )}^{2} + {( \frac{1}{4} )}^{2} + {( \frac{1}{4} )}^{2} ] \} \times \frac{4}{5} + \{ 1 - {( \frac{1}{1} )}^{2} \} \times \frac{1}{5} \} \times 0.02 = 0.01 \qquad Equation 16$$

For example, after sorting the physiological measurement data according to total cholesterol, the fourth cutting point is between 235 and 285. In the physiological measurement data whose total cholesterol are 150, 187, 201 and 235 (e.g., patients No. 1, No. 4, No. 3, and No. 5), the diseases they correspond to are fatty liver, hypertension, and diabetes, thus the probability of occurrence of fatty liver is ¼, the probability of occurrence of hypertension is ¼, and the probability of occurrence of diabetes is 2/4. Therefore, the left branch of the fourth cutting point is

$$1 - [ {( \frac{2}{4} )}^{2} + {( \frac{1}{4} )}^{2} + {( \frac{1}{4} )}^{2} ]$$

multiplying by ⅘ (4 of 5 data). Similarly, in the physiological measurement data whose total cholesterol is 285 (e.g. patient No. 2), the disease it corresponds to is atherosclerosis, thus the probability is 1/1. Therefore, the right branch is

$$1 - {( \frac{1}{1} )}^{2}$$

multiplying by ⅕ (1 of 5 data). According to the result of Equation 16, it can be obtained that the value of the fourth cutting point sorted by total cholesterol is 0.01.

After that, the method of the present disclosure sorts the data of patients No. 1 to 5 as (4, 3, 1, 5, 2) according to white blood cells, that is, the sorting of white blood cells of patients No. 1 to 5 is (4.38, 8.51, 15.3, 18.1, 20.8). The method of the present disclosure first calculates the first cutting point according to white blood cells, that is, the first cutting point for white blood cells<((4.38+8.51)/2), as shown in Equation 17 below.

$$Gini ( D ) \times \vert r \vert = \{ \{ 1 - [ {( \frac{1}{1} )}^{2} ] \} \times \frac{1}{5} + \{ 1 - [ {( \frac{2}{4} )}^{2} + {( \frac{1}{4} )}^{2} + {( \frac{1}{4} )}^{2} ] \} \times \frac{4}{5} \} \times 1 = 0.5 \qquad Equation 17$$

For example, after sorting the physiological measurement data according to white blood cells, the first cutting point is between 4.38 and 8.51. In the physiological measurement data whose white blood cells is 4.38 (e.g., patient No. 4), the disease it corresponds to is fatty liver, thus the probability is 1/1. Therefore, the left branch of the first cutting point is

$$1 - [ {( \frac{1}{1} )}^{2} ]$$

multiplying by ⅕ (1 of 5 data). Similarly, in the physiological measurement data whose white blood cells are 8.51, 15.3, 18.1 and 20.8 (e.g. patients No. 1˜3 and No. 5), the diseases they correspond to are diabetes, atherosclerosis and hypertension, thus the probability of occurrence of diabetes is 2/4, the probability of occurrence of atherosclerosis is ¼, and the probability of occurrence of hypertension is ¼. Therefore, the right branch is

$$1 - [ {( \frac{2}{4} )}^{2} + {( \frac{1}{4} )}^{2} + {( \frac{1}{4} )}^{2} ]$$

multiplying by ⅘ (4 of 5 data). According to the result of Equation 17, it can be obtained that the value of the first cutting point sorted by white blood cells is 0.5.

Then, the method of the present disclosure calculates the next cutting point according to white blood cells, that is, the second cutting point for white blood cells <((8.51+15.3)/2), as shown in Equation 18 below.

$$Gini ( D ) \times \vert r \vert = \{ \{ 1 - [ {( \frac{1}{2} )}^{2} + {( \frac{1}{2} )}^{2} ] \} \times \frac{2}{5} + \{ 1 - [ {( \frac{2}{3} )}^{2} + {( \frac{1}{3} )}^{2} ] \} \times \frac{3}{5} \} \times 1 = 0.4667 \qquad Equation 18$$

For example, after sorting the physiological measurement data according to white blood cells, the second cutting point is between 8.51 and 15.3. In the physiological measurement data whose white blood cells are 4.38 and 8.51 (e.g., patients No. 4 and No. 3), the diseases they correspond to are fatty liver and hypertension, thus the probability is ½ each. Therefore, the left branch of the second cutting point is

$$1 - [ {( \frac{1}{2} )}^{2} + {( \frac{1}{2} )}^{2} ]$$

multiplying by ⅖ (2 of 5 data). Similarly, in the physiological measurement data whose white blood cells are 15.3, 18.1 and 20.8 (e.g., patients No. 1, No. 5 and No. 2), the diseases they correspond to are diabetes and atherosclerosis, thus the probability of occurrence of diabetes is ⅔, and the probability of occurrence of atherosclerosis is ⅓. Therefore, the right branch is

$$1 - [ {( \frac{2}{3} )}^{2} + {( \frac{1}{3} )}^{2} ]$$

multiplying by ⅗ (3 of 5 data). According to the result of Equation 18, it can be obtained that the value of the second cutting point sorted by white blood cells is 0.4667.

Then, the method of the present disclosure calculates the next cutting point according to white blood cells, that is, the third cutting point for white blood cells<((15.3+18.1)/2), as shown in Equation 19 below.

$$Gini ( D ) \times \vert r \vert = \{ \{ 1 - [ {( \frac{1}{3} )}^{2} + {( \frac{1}{3} )}^{2} + {( \frac{1}{3} )}^{2} ] \} \times \frac{3}{5} + \{ 1 - [ {( \frac{1}{2} )}^{2} + {( \frac{1}{2} )}^{2} ] \} \times \frac{2}{5} \} \times 0.9987 = 0.599 \qquad Equation 19$$

For example, after sorting the physiological measurement data according to white blood cells, the third cutting point is between 15.3 and 18.1. In the physiological measurement data whose white blood cells are 4.38, 8.51 and 15.3 (e.g., patients No. 4, No. 3 and No. 1), the diseases they correspond to are all different (such as fatty liver, hypertension, and diabetes), thus the probability is ⅓ each. Therefore, the left branch of the third cutting point is

$$1 - [ {( \frac{1}{3} )}^{2} + {( \frac{1}{3} )}^{2} + {( \frac{1}{3} )}^{2} ]$$

multiplying by 3/5 (3 of 5 data). Similarly, in the physiological measurement data whose white blood cells are 18.1 and 20.8 (e.g., patients No. 5 and No. 2), the diseases they correspond to are different (such as diabetes and atherosclerosis), thus the probability is 1/2 each. Therefore, the right branch is

$$1 - [ {( \frac{1}{2} )}^{2} + {( \frac{1}{2} )}^{2} ]$$

multiplying by ⅖ (2 of 5 data). According to the result of Equation 19, it can be obtained that the value of the third cutting point sorted by white blood cells is 0.599.

Then, the method of the present disclosure calculates the next cutting point according to white blood cells, that is, the fourth cutting point for white blood cells<((18.1+20.8)/2), as shown in Equation 20 below.

$$Gini ( D ) \times \vert r \vert = \{ \{ 1 - [ {( \frac{2}{4} )}^{2} + {( \frac{1}{4} )}^{2} + {( \frac{1}{4} )}^{2} ] \} \times \frac{4}{5} + \{ 1 - {( \frac{1}{1} )}^{2} \} \times \frac{1}{5} \} \times 0.9832 = 0.4916 \qquad Equation 20$$

For example, after sorting the physiological measurement data according to white blood cells, the fourth cutting point is between 18.1 and 20.8. In the physiological measurement data whose white blood cells are 4.38, 8.51, 15.3 and 18.1 (e.g., patients No. 4, No. 3, No. 1, and No. 5), the diseases they correspond to are fatty liver, hypertension, and diabetes, thus the probability of occurrence of fatty liver is ¼, the probability of occurrence of hypertension is ¼, and the probability of occurrence of diabetes is 2/4. Therefore, the left branch of the fourth cutting point is

$$1 - [ {( \frac{2}{4} )}^{2} + {( \frac{1}{4} )}^{2} + {( \frac{1}{4} )}^{2} ]$$

multiplying by 4/5 (4 of 5 data). Similarly, in the physiological measurement data whose white blood cells is 20.8 (e.g. patient No. 2), the disease it corresponds to is atherosclerosis, thus the probability is 1/1. Therefore, the right branch is

$$1 - {( \frac{1}{1} )}^{2}$$

multiplying by ⅕ (1 of 5 data). According to the result of Equation 20, it can be obtained that the value of the fourth cutting point sorted by white blood cells is 0.4916.

The method of the present disclosure sorts the data of patients No. 1 to 5 as (4, 3, 1, 5, 2) according to blood sugar, that is, the sorting of blood sugar of patients No. 1 to 5 is (100, 125, 131, 185, 201). The method of the present disclosure first calculates the first cutting point according to blood sugar, that is, the first cutting point for blood sugar<((100+125)/2), as shown in Equation 21 below.

$$Gini ( D ) \times \vert r \vert = \{ \{ 1 - [ {( \frac{1}{1} )}^{2} ] \} \times \frac{1}{5} + \{ 1 - [ {( \frac{2}{4} )}^{2} + {( \frac{1}{4} )}^{2} + {( \frac{1}{4} )}^{2} ] \} \times \frac{4}{5} \} \times 1 = 0.5 \qquad Equation 21$$

For example, after sorting the physiological measurement data according to blood sugar, the first cutting point is between 100 and 125. In the physiological measurement data whose blood sugar is 100 (e.g., patient No. 3), the disease it corresponds to is hypertension, thus the probability is 1/1. Therefore, the left branch of the first cutting point is

$$1 - [ {( \frac{1}{1} )}^{2} ]$$

multiplying by ⅕ (1 of 5 data). Similarly, in the physiological measurement data whose blood sugar are 125, 131, 185 and 201 (e.g. patients No. 2, No. 4, No. 5 and No. 1), the diseases they correspond to are diabetes, atherosclerosis and hypertension, thus the probability of occurrence of diabetes is 2/4, the probability of occurrence of atherosclerosis is ¼, and the probability of occurrence of hypertension is ¼. Therefore, the right branch is

$$1 - [ {( \frac{2}{4} )}^{2} + {( \frac{1}{4} )}^{2} + {( \frac{1}{4} )}^{2} ]$$

multiplying by ⅘ (4 of 5 data). According to the result of Equation 21, it can be obtained that the value of the first cutting point sorted by blood sugar is 0.5.

Then, the method of the present disclosure calculates the next cutting point according to blood sugar, that is, the second cutting point for blood sugar<(125+131)/2), as shown in Equation 22 below.

$$Gini ( D ) \times \vert r \vert = \{ \{ 1 - [ {( \frac{1}{2} )}^{2} + {( \frac{1}{2} )}^{2} ] \} \times \frac{2}{5} + \{ 1 - [ {( \frac{2}{3} )}^{2} + {( \frac{1}{3} )}^{2} ] \} \times \frac{3}{5} \} \times 1 = 0.4667 \qquad Equation 22$$

For example, after sorting the physiological measurement data according to blood sugar, the second cutting point is between 125 and 131. In the physiological measurement data whose blood sugar are 100 and 125 (e.g., patients No. 3 and No. 2), the diseases they correspond to are fatty liver and atherosclerosis, thus the probability is ½ each. Therefore, the left branch of the second cutting point is

$$1 - [ {( \frac{1}{2} )}^{2} + {( \frac{1}{2} )}^{2} ]$$

multiplying by ⅖ (2 of 5 data). Similarly, in the physiological measurement data whose blood sugar are 131, 185 and 201 (e.g., patients No. 4, No. 5 and No. 1), the diseases they correspond to are diabetes and fatty liver, thus the probability of occurrence of diabetes is ⅔, and the probability of occurrence of fatty liver is ⅓. Therefore, the right branch is

$$1 - [ {( \frac{2}{3} )}^{2} + {( \frac{1}{3} )}^{2} ]$$

multiplying by ⅗ (3 of 5 data). According to the result of Equation 22, it can be obtained that the value of the second cutting point sorted by blood sugar is 0.4667.

Then, the method of the present disclosure calculates the next cutting point according to blood sugar, that is, the third cutting point for blood sugar<((131+185)/2), as shown in Equation 23 below.

$$Gini ( D ) \times \vert r \vert = \{ \{ 1 - [ {( \frac{1}{3} )}^{2} + {( \frac{1}{3} )}^{2} + {( \frac{1}{3} )}^{2} ] \} \times \frac{3}{5} + \{ 1 - [ {( \frac{2}{2} )}^{2} ] \} \times \frac{2}{5} \} \times 0.1825 = 0.073 \qquad Equation 23$$

For example, after sorting the physiological measurement data according to blood sugar, the third cutting point is between 131 and 185. In the physiological measurement data whose blood sugar are 100, 125 and 131 (e.g., patients No. 3, No. 2 and No. 4), the diseases they correspond to are all different (such as fatty liver, hypertension, and atherosclerosis), thus the probability is ⅓ each. Therefore, the left branch of the third cutting point is

$$1 - [ {( \frac{1}{3} )}^{2} + {( \frac{1}{3} )}^{2} + {( \frac{1}{3} )}^{2} ]$$

multiplying by ⅗ (3 of 5 data). Similarly, in the physiological measurement data whose blood sugar are 185 and 201 (e.g., patients No. 5 and No. 1), the disease they correspond to is the same (such as diabetes), thus the probability is 2/2. Therefore, the right branch is

$$1 - [ {( \frac{2}{2} )}^{2} ]$$

multiplying by ⅖ (2 of 5 data). According to the result of Equation 23, it can be obtained that the value of the third cutting point sorted by blood sugar is 0.073.

Then, the method of the present disclosure calculates the next cutting point according to blood sugar, that is, the fourth cutting point for blood sugar<((185+201)/2), as shown in Equation 24 below.

$$Gini ( D ) \times \vert r \vert = \{ \{ 1 - [ {( \frac{1}{4} )}^{2} + {( \frac{1}{4} )}^{2} + {( \frac{1}{4} )}^{2} + {( \frac{1}{4} )}^{2} ] \} \times \frac{4}{5} + \{ 1 - {( \frac{1}{1} )}^{2} \} \times \frac{1}{5} \} \times 0.6747 = 0.4048 \qquad Equation 24$$

For example, after sorting the physiological measurement data according to blood sugar, the fourth cutting point is between 185 and 201. In the physiological measurement data whose blood sugar are 100, 125, 131 and 185 (e.g., patients No. 3, No. 2, No. 4, and No. 5), the diseases they correspond to are all different (such as fatty liver, hypertension, atherosclerosis and diabetes), thus the probability of occurrence of fatty liver is ¼, the probability of occurrence of hypertension is ¼, he probability of occurrence of atherosclerosis is ¼, and the probability of occurrence of diabetes is ¼. Therefore, the left branch of the fourth cutting point is

$$1 - [ {( \frac{1}{4} )}^{2} + {( \frac{1}{4} )}^{2} + {( \frac{1}{4} )}^{2} + {( \frac{1}{4} )}^{2} ]$$

multiplying by ⅘ (4 of 5 data). Similarly, in the physiological measurement data whose blood sugar is 201 (e.g. patient No. 1), the disease it corresponds to is diabetes, thus the probability is 1/1. Therefore, the right branch is

$$1 - {( \frac{1}{1} )}^{2}$$

multiplying by ⅕ (1 of 5 data). According to the result of Equation 24, it can be obtained that the value of the fourth cutting point sorted by blood sugar is 0.4048. So far, the method of present disclosure has complete step S104 in FIG. 1.

In step S104, the method of present disclosure obtains the value of the cutting point sorted by gender as 0.6. The method of present disclosure obtains the value of the first, second, third and fourth cutting points sorted by BMI as 0.6, 0.6, 0.054, and 0.158. The method of present disclosure obtains the value of the first, second, third and fourth cutting points sorted by uric acid as 0.5, 0.4667, 0.589, and 0.4938. The method of present disclosure obtains the value of the first, second, third and fourth cutting points sorted by total cholesterol as 0.6, 0.6, 0.4944, and 0.01. The method of present disclosure obtains the value of the first, second, third and fourth cutting points sorted by white blood cells as 0.5, 0.4667, 0.599, and 0.4916. The method of present disclosure obtains the value of the first, second, third and fourth cutting points sorted by blood sugar as 0.5, 0.4667, 0.073, and 0.4048.

After that, in step S106 in FIG. 1, the method of present disclosure sets the cutting point with the smallest value from all the above-mentioned cutting points as a branch node of the decision tree of the present disclosure. In other words, since the value of the fourth cutting point sorted by total cholesterol is 0.01, which is the smallest among all the above-mentioned cutting points, the present disclosure set the fourth cutting point sorted by total cholesterol (e.g., total cholesterol<((235+285)/2=260) as the branch node of the decision tree.

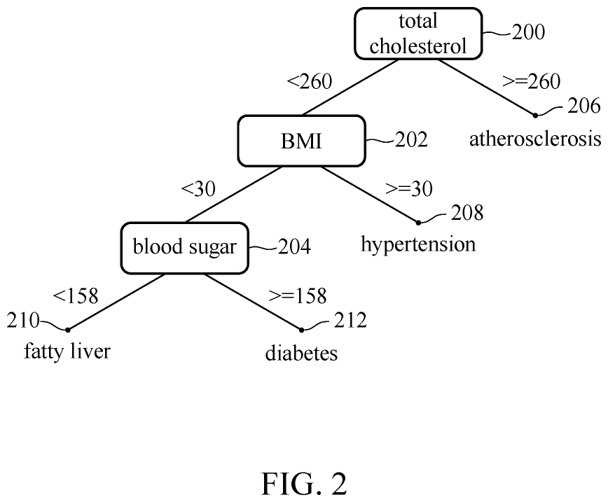

FIG. 2 is a schematic diagram of the decision tree in accordance with some embodiments of the disclosure. Continuing the content of the previous paragraph, the method of present disclosure can obtain that the value of the fourth cutting point sorted by total cholesterol is the smallest value (0.01), so a branch node 200 is set as total cholesterol. The left branch of the branch node 200 is the physiological measurement data with total cholesterol<260 (for example, the data of patients No. 1 and No. 3˜5), and the right branch of the branch node 200 is the physiological measurement data with total cholesterol>=260 (for example, the data of patient No. 2). Then, the branching according to the methods in the previous paragraphs is continued, and the decision tree in FIG. 2 can be obtained. Since the right branch of the branch node 200 leaves one of the physiological measurement data corresponding to atherosclerosis (for example, the data of patient No. 2), the number of physiological measurement data included in a branch node 206 (for example, one data) is less than or equal to a preset number of physiological measurement data (for example, one data, that is, the data of patient No. 2) corresponding to a disease (for example, atherosclerosis). Therefore, the method of present disclosure sets the branch node 206 as a terminal branch node (e.g., the branching of the branch node 206 cannot be continued), and sets atherosclerosis in the branch node 206.

Since the left branch of the branch node 200 (total cholesterol<260) leaves 4 of the physiological measurement data (data of patients No. 1, No. 2, No. 3 and No. 5), the method of present disclosure also executes steps S104 and S106, and obtains that the third cutting point sorted by BMI (BMI<((28+32)/2)=30) has the smallest value, so a branch node 202 is set as BMI. The left branch of the branch node 202 is the physiological measurement data with BMI<30 (for example, the data of patients No. 1, No. 4 and No. 5), and the right branch of the branch node 202 is the physiological measurement data with BMI>=30 (for example, the data of patient No. 3). In step S106, since the right branch of the branch node 202 leaves one of the physiological measurement data corresponding to hypertension (for example, the data of patient No. 3), the number of physiological measurement data included in a branch node 208 (for example, one data) is less than or equal to a preset number of physiological measurement data (for example, one data, that is, the data of patient No. 3) corresponding to a disease (for example, hypertension). Therefore, the method of present disclosure sets the branch node 208 as a terminal branch node (e.g., the branching of the branch node 208 cannot be continued), and sets hypertension in the branch node 208.

Since the left branch of the branch node 202 (BMI<30) leaves 3 of the physiological measurement data (data of patients No. 1, No. 4 and No. 5), the method of present disclosure also executes steps S104 and S106, and obtains that the first cutting point sorted by blood sugar (blood sugar<((131+185)/2)=158) has the smallest value, so a branch node 204 is set as blood sugar. The left branch of the branch node 204 is the physiological measurement data with blood sugar<158 (for example, the data of patient No. 4), and the right branch of the branch node 204 is the physiological measurement data with blood sugar>=158 (for example, the data of patients No. 1 and No. 5). In step S106, since the right branch of the branch node 204 leaves two of the physiological measurement data corresponding to hypertension (for example, the data of patients No. 1 and No. 5), the number of physiological measurement data included in the branch node 208 (for example, two data) is less than or equal to a preset number of physiological measurement data (for example, two data, that is, the data of patients No. 1 and No. 5) corresponding to a disease (for example, diabetes). Therefore, the method of present disclosure sets a branch node 212 as a terminal branch node (e.g., the branching of the branch node 212 cannot be continued), and sets diabetes in the branch node 212.

Moreover, since the left branch of the branch node 204 leaves one of the physiological measurement data corresponding to fatty liver (for example, the data of patient No. 4), the number of physiological measurement data included in the branch node 210 (for example, one data) is less than or equal to a preset number of physiological measurement data (for example, one data, that is, the data of patient No. 4) corresponding to a disease (for example, fatty liver). Therefore, the method of present disclosure sets a branch node 210 as a terminal branch node (e.g., the branching of the branch node 210 cannot be continued), and sets fatty liver in the branch node 210. To put it simply, the branch nodes 200, 202 and 204 are obtained by being determined as “no” in step S106 in FIG. 1, and the branch nodes 206, 208, 210 and 212 (terminal branch nodes) are obtained by being determined as “yes” in step S106 in FIG. 1.

In step S108, Akaike Information Criterion (AIC) is a criterion used to check whether the decision tree in FIG. 2 is overfitting. In some embodiments, the AIC is as follows.

AIC=−2×1+2×(k+1) Equation 25

In Equation 25, 1 is a likelihood function, and k is the number of parameters. In some embodiments, the method of present disclosure further calculates the correct rate of each terminal branch node (e.g., the branch nodes 206, 208, 210, and 212 in FIG. 2) corresponding to different diseases in the decision tree in FIG. 2.

The method of the present disclosure inputs the following three prediction data in Table 2 into the decision tree of FIG. 2 to obtain the prediction result of patient A, which is disease 1, the prediction result of patient B, which is disease 2, and the prediction result of patient C, which is disease 3.

TABLE 2

| | | | | Total | White | | |
|---|---|---|---|---|---|---|---|
| | | | Uric | choles- | blood | Blood | Prediction |
| Patient | Gender | BMI | acid | terol | cells | sugar | result |
| A | F | 30 | 4.5 | 200 | 13.1 | 189 | Disease 1 |
| B | M | 20 | 4.7 | 203 | 15.7 | 161 | Disease 2 |
| C | F | 25 | 7.8 | 195 | 25.3 | 155 | Disease 3 |

Table 3 is the judgment of disease characteristics by the decision tree in FIG. 2.

TABLE 3

| | Total | Total | | | Blood | Blood |
|---|---|---|---|---|---|---|
| | cholesterol < | cholesterol >= | BMI < | BMI >= | sugar < | sugar >= |
| Disease | 260 | 260 | 30 | 30 | 158 | 158 |
| Diabetes | match | mismatch | match | mismatch | mismatch | match |
| Fatty liver | match | mismatch | match | mismatch | match | mismatch |
| Hypertension | match | mismatch | mismatch | match | NA | NA |
| Atherosclerosis | mismatch | match | NA | NA | NA | NA |

According to Table 3, the method of present disclosure can obtain that total cholesterol for patient A is lower than 260, and the BMI for patient A is higher than or equal to 30, thus patient A may suffer from hypertension. And so on, patient B may suffer from diabetes, and patient C may suffer from fatty liver. The above result can be used as auxiliary conditions for doctors' diagnosis.

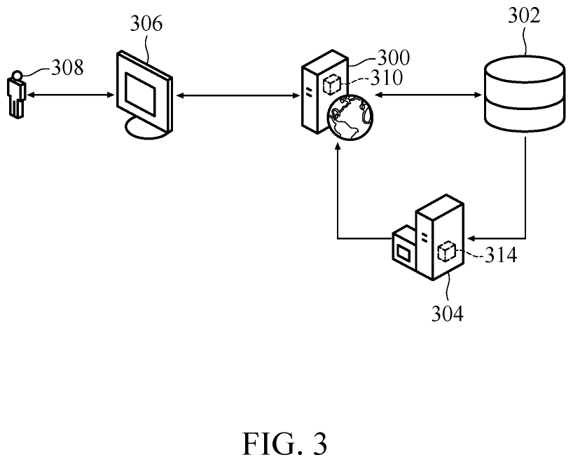

FIG. 3 is a schematic diagram of an electronic system to establish a decision tree for disease prediction in accordance with some embodiments of the disclosure. As shown in FIG. 3, the electronic system includes a network server 300, a data base 302, and a computing server 304. The network server 300 includes a processor 310. The computing server 304 includes a processor 314. In some embodiments, the processor 310 of the network server 300 executes step S100 in FIG. 1. In some embodiments, the physiological measurement data in step S100 corresponding to different diseases come from a computer 306 in the hospital. The physiological measurement data in step S100 come from the measurements and diagnosis results of doctor 308 on different patients. The data base 302 stores the physiological measurement data. The processor 314 of the computing server 304 executes steps S104, S106, and S108 in FIG. 1. In some embodiments, the processor 314 of the computing server 304 can send its disease prediction results to the network server 300 for publishing its disease prediction results to everyone.

The present disclosure also provides a computer program product to establish a decision tree (for example, the decision tree in FIG. 2) for disease prediction. The computer program product is applied to an electronic system (for example, the electronic system in FIG. 3) having a first processor (for example, the processor 310 in FIG. 3), a second processor (for example, the processor 314 in FIG. 3), and a data base (for example, the data base 302 in FIG. 3). The computer program product of the present disclosure includes a receiving instruction, a storing instruction, a reading instruction, a classifying instruction, a calculating instruction, a branching instruction, and a pruning instruction. In some embodiments, the receiving instruction enables the processor 310 to execute step S100 in FIG. 1. The storing instruction enables the data base 302 to store the physiological measurement data corresponding to different diseases in step S100. The reading instruction enables the processor 314 to obtain the physiological measurement data from the data base 302. The classifying instruction enables the processor 314 to execute step S102 in FIG. 1.

The calculating instruction enables the processor 314 to execute step S104 in FIG. 1. The branching instruction enables the processor 314 to execute step S106 in FIG. 1. The pruning instruction enables the processor 314 to execute step S108 in FIG. 1. After the processor 310 finishes executing the receiving instruction, the data base 302 finishes executing the storing instruction, and the processor finishes executing the reading instruction, the classifying instruction, the calculating instruction, the branching instruction, and the pruning instruction, the establishment of the decision tree in FIG. 2 is completed (corresponding to step S108 in FIG. 1).

The more the physiological measurement data from the hospital, the more accurate the prediction results obtained by the method, electronic system, and computer program product of the present disclosure for establishing a decision tree for disease prediction. The method, electronic system, and computer program product of the present disclosure can assist doctors in medical diagnosis, and give preventive medication in advance according to prediction results. The method, electronic system, and computer program product of the present disclosure can calculate the data of each terminal branch node of the decision tree to obtain the probability of a single disease, which can improve the accuracy of more disease predictions.

The embodiments of the present disclosure are disclosed above, but they are not used to limit the scope of the present disclosure. A person skilled in the art can make some changes and retouches without departing from the spirit and scope of the embodiments of the present disclosure. Therefore, the scope of protection in the present disclosure shall be deemed as defined by the scope of the attached claims.

What is claimed is:

1. A method for establishing a decision tree for disease prediction, comprising:
receiving a plurality of physiological measurement data corresponding to different diseases;
classifying the physiological measurement data corresponding to the purpose;
calculating at least one cutting point of the physiological measurement data;
branching the decision tree corresponding to the at least one cutting point; and
pruning the decision tree to complete the establishment of the decision tree.

2. The method as claimed in claim 1, wherein the step of calculating the at least one cutting point of the physiological measurement data comprises:
calculating a value of the at least one cutting point of the physiological measurement data by using a specific function associated with the physiological measurement data and the absolute value of a correlation coefficient associated with the physiological measurement data.

3. The method as claimed in claim 2, wherein the step of branching the decision tree corresponding to the at least one cutting point comprises:
setting the at least one cutting point with the smallest value as a branch node of the decision tree; and
determining whether the step of branching can be continued or not.

4. The method as claimed in claim 1, wherein the step of pruning the decision tree to complete the establishment of the decision tree comprises:
pruning the decision tree by using an Akaike Information Criterion (AIC).

5. The method as claimed in claim 2, wherein the step of classifying the physiological measurement data corresponding to the purpose comprises:
classifying the physiological measurement data as classification data when the physiological measurement data are used for the estimation of the probability of occurrence of different diseases.

6. The method as claimed in claim 5, wherein when the physiological measurement data are classified as the classification data, the specific function is a Gini coefficient formula;
the Gini coefficient formula is as follows:
Gini(D)=Σᵢ₌₁ⁿp(xᵢ)×(1−p(xᵢ))=1−Σᵢ₌₁ⁿp(xᵢ)²
wherein xᵢ is the data corresponding to a disease among the physiological measurement data; p(xᵢ) is the probability of occurrence of the data corresponding to the disease among the physiological measurement data; and n is the number of disease types corresponding to the physiological measurement data.

7. The method as claimed in claim 6, wherein the correlation coefficient is as follows:
$$r ( i ) = \frac{{\sum }_{j = 1}^{n} ( {x}_{j} - \overline{x} ) ( {y}_{j} - \overline{y} )}{\sqrt{{\sum }_{j = 1}^{n} {( {x}_{j} - \overline{x} )}^{2}} \sqrt{{\sum }_{j = 1}^{n} {( {y}_{j} - \overline{y} )}^{2}}} ;$$
wherein i is the physiological measurement data; n is number of physiological measurement data; xᵢ is an independent variable and represents the physiological measurement data; x̅ is the mean of independent variables and represents the mean of the physiological measurement data; yⱼ is a dependent variable and represents the value corresponding to a disease; and y̅ is the mean of dependent variables and represents the mean of the value corresponding to the disease.

8. The method as claimed in claim 1, wherein the physiological measurement data comprises gender, Body Mass Index (BMI), uric acid, total cholesterol, white blood cells, and blood sugar.

9. The method as claimed in claim 7, wherein the value of the at least one cutting point of the physiological measurement data is equal to Gini(D)×|r(i)|.

10. The method as claimed in claim 4, wherein the AIC is as follows:
AIC=−2×1+2×(k+1)
wherein 1 is a likelihood function, and k is the number of parameters.

11. The method as claimed in claim 1, further comprising:
calculating the correct rate of each terminal branch of the decision tree corresponding to the different diseases.

12. The method as claimed in claim 3, wherein in response to determine whether the step of branching can be continued or not, the method comprises:
repeating the step of calculating the value of the at least one cutting point of the physiological measurement data and the step of setting the at least one cutting point with the smallest value as the branch node of the decision tree, until the step of branching cannot be continued; or
repeating the step of calculating the value of the at least one cutting point of the physiological measurement data and the step of setting the at least one cutting point with the smallest value as the branch node of the decision tree, until the number of physiological measurement data included in the branch node is less than or equal to a preset number of physiological measurement data corresponding to each disease.

13. The method as claimed in claim 8, wherein in response to determine whether the step of branching can be continued or not, the method comprises:
sorting the physiological measurement data according to gender from female to male;
sorting the physiological measurement data according to BMI from low to high;
sorting the physiological measurement data according to uric acid from low to high;
sorting the physiological measurement data according to total cholesterol from least to most;
sorting the physiological measurement data according to the number of white blood cells from least to most; and
sorting the physiological measurement data according to blood sugar from low to high.

14. The method as claimed in claim 13, wherein in response to determine whether the step of branching can be continued or not, the method comprises:
calculating the product between the specific function and the absolute value of the correlation coefficient according to the result of sorting by gender, BMI, uric acid, total cholesterol, white blood cells, and blood sugar of the physiological measurement data.

15. An electronic system to establish a decision tree for disease prediction, comprising:
a first processor, configured to receive a plurality of physiological measurement data corresponding to different diseases from a hospital;
a data base, configured to store the physiological measurement data; and
a second processor, configured to obtain the physiological measurement data from the data base to execute the following steps:
classifying the physiological measurement data corresponding to the purpose;
calculating at least one cutting point of the physiological measurement data;
branching the decision tree corresponding to the at least one cutting point; and
pruning the decision tree to complete the establishment of the decision tree.

16. The electronic system as claimed in claim 15, wherein calculating the at least one cutting point of the physiological measurement data using the second processor comprises:
using the second processor to calculate the value of the at least one cutting point of the physiological measurement data by using a specific function associated with the physiological measurement data and the absolute value of the correlation coefficient associated with the physiological measurement data.

17. The electronic system as claimed in claim 16, wherein when the physiological measurement data are used for the estimation of the probability of occurrence of different diseases, the second processor classifies the physiological measurement data as classification data.

18. The electronic system as claimed in claim 17, wherein when the second processor classifies the physiological measurement data as the classification data, the specific function is a Gini coefficient formula;
the Gini coefficient formula is as follows:
Gini(D)=Σᵢ₌₁ⁿp(xᵢ)×(1−p(xᵢ))=1−Σᵢ₌₁ⁿp(xᵢ)²
wherein xᵢ is the data corresponding to a disease among the physiological measurement data; p(xᵢ) is the probability of occurrence of the data corresponding to the disease among the physiological measurement data; and n is the number of disease types corresponding to the physiological measurement data.

19. The electronic system as claimed in claim 18, wherein the correlation coefficient is as follows:
$$r ( i ) = \frac{{\sum }_{j = 1}^{n} ( {x}_{j} - \overline{x} ) ( {y}_{j} - \overline{y} )}{\sqrt{{\sum }_{j = 1}^{n} {( {x}_{j} - \overline{x} )}^{2}} \sqrt{{\sum }_{j = 1}^{n} {( {y}_{j} - \overline{y} )}^{2}}} ;$$
wherein i is one of the physiological measurement data; n is the number of physiological measurement data; xⱼ is an independent variable and represents the physiological measurement data; x̅ is the mean of independent variables and represents the mean of the physiological measurement data; yⱼ is a dependent variable and represents the value corresponding to a disease; and y̅ is the mean of dependent variables and represents the mean of the value corresponding to the disease.

20. The electronic system as claimed in claim 19, wherein the value of the at least one cutting point of the physiological measurement data is equal to Gini(D)×|r(i)|.

21. A computer program product to establish a decision tree for disease prediction, applied to an electronic system having a first processor, a second processor, and a data base, comprising:
a receiving instruction, enabling the first processor to receive a plurality of physiological measurement data corresponding to different diseases from a hospital;
a storing instruction, enabling the data base to store the physiological measurement data;
a reading instruction, enabling the second processor to obtain the physiological measurement data from the data base;
a classifying instruction, enabling the second processor to classify the physiological measurement data corresponding to the purpose;
a calculating instruction, enabling the second processor to calculate at least one cutting point of the physiological measurement data;
a branching instruction, enabling the second processor to branch the decision tree corresponding to the at least one cutting point; and
a pruning instruction, enabling the second processor to prune the decision tree;
wherein after the first processor finishes the receiving instruction, the data base finishes executing the storing instruction, and the second processor finishes the reading instruction, the classifying instruction, the calculating instruction, the branching instruction, and the pruning instruction, the establishment of the decision tree is completed.
